(19) United States
(12) Patent Application Publication
(10) Pub. No.: US 2023/0323455 A1
(43) Pub. Date: Oct. 12, 2023

(54) METHODS FOR ASSESSING THE SEVERITY AND PROGRESSION OF SARS-COV2 INFECTIONS USING CELL-FREE DNA

(71) Applicant: CORNELL UNIVERSITY, Ithaca, NY (US)

(72) Inventors: Iwijn DE VLAMINCK, Ithaca, NY (US); Alexandre Pellan CHENG, Ithaca, NY (US); Matthew Pellan CHENG, Montreal (CA); Donald Cuong VINH, Montreal-West (CA); Wei GU, Menlo Park, CA (US); Charles CHIU, San Francisco, CA (US)

(21) Appl. No.: 18/017,165

(22) PCT Filed: Jul. 23, 2021
(86) PCT No.: PCT/US2021/042875

§ 371 Date: Jan. 20, 2023

Related U.S. Application Data
(60) Provisional application No. 63/056,249, filed on Jul. 24, 2020.

(51) Int. Cl. C12Q 1/70; A61K 31/573; A61K 31/664

(52) U.S. Cl. CPC C12Q 1/70; A61K 31/573; A61K 31/664; C12Q 2600/106; C12Q 2600/118; C12Q 2600/154; C12Q 2600/156; C12Q 2600/158

(57) ABSTRACT

The present disclosure is directed to methods for assessing the severity and progression of a SARS-CoV2 infection in a subject using cell-free DNA (cfDNA). The present disclosure is further directed to methods for assessing tissue damage as a result of SARS-CoV2 infection. In addition, the present disclosure provides methods for treating a SARS-CoV2 infection in a subject. Finally, the disclosure also provides methods for detecting a microbial co-infection of a subject suffering from a SARS-CoV2 infection.

### CROSS REFERENCE TO RELATED APPLICATION

This application claims the benefit of priority from U.S. Provisional Application No. 63/056,249, filed Jul. 24, 2020, the entire contents of which are incorporated herein by reference.

### STATEMENT REGARDING FEDERALLY SPONSORED RESEARCH OR DEVELOPMENT

This invention was made with government support under Grant Nos. 1DP2AI138242, R01AI146165, 1R01AI151059, R33-AI129455and K08-CA230156, awarded by the National Institutes of Health. The government has certain rights in the invention.

### BACKGROUND

The Coronavirus Disease-19 (COVID-19) pandemic is a major global health crisis. COVID-19 is a complex disease with diverse clinical features, ranging from asymptomatic infection to acute respiratory distress syndrome (ARDS) and multi-organ dysfunction. There is an urgent need for improved understanding of the pathogenesis of COVID-19.

Autopsy studies indicate a broad organotropism for the SARS-CoV-2 virus beyond the lungs. Detection of the virus in the kidneys, heart, liver, brain, and blood of many patients has been reported. The significant viral burden in the kidney seen in some patients may help explain the increased risk of acute kidney injury in patients with COVID-19. Damage to endothelial cells on the other hand may explain the coagulopathic state and increased risk for thrombotic events associated with COVID-19.

Initial reports have primarily described COVID-19 as a disease affecting tissues expressing ACE-2. However, there are emerging data that SARS-CoV-2 infection may also be accompanied by hematological derangements. In addition, a dysregulated immune response to SARS-CoV-2 is reported, contributing to the development of ARDS, systemic tissue injury, and multi-organ failure. A strong association between increased cytokine profiles and the severe deterioration of some patients has been observed. In children, a multisystem inflammatory syndrome linked to recent SARS-CoV-2 infection is reported. Given the disparate clinical manifestations and potential complications of COVID-19, there is an urgent need for tests that can quantify injury to multiple tissues simultaneously to monitor patients, analyze disease pathogenesis, predict clinical outcomes, and guide clinical management in patients with COVID-19.

Since the advent of cfDNA based noninvasive prenatal testing, myriad applications of cfDNA in diagnostic medicine have been established. These short fragments of circulating DNA are the debris of dead cells from across the body. The value of cfDNA as a quantitative marker of tissue and organ injury was first recognized in solid-organ transplantation, where the level of transplant donor derived cfDNA in the blood is now widely used as a marker of transplant rejection. More recently, several approaches have been developed to quantify the tissues-of-origin of cfDNA and thus monitor injury to any cell, tissue or organ type. This is achieved by profiling epigenetic marks within cfDNA by quantitative molecular measurement technologies such as DNA sequencing.

### SUMMARY OF THE DISCLOSURE

An aspect of the disclosure is directed to a method for assessing the severity and progression of a SARS-CoV2 infection in a subject, comprising: measuring the amount of total cell-free DNA (cfDNA) molecules in a biological sample from the subject, wherein an increased level of total cfDNA molecules from the sample as compared to a control level is indicative of increased severity and disease progression of SARS-CoV2 infection in the subject.

Another aspect of the disclosure is directed to a method for assessing the likelihood to benefit from a selected clinical intervention given to a subject suffering from a SARS-CoV2 infection, comprising: measuring the amount of total cell-free DNA (cfDNA) molecules in a biological sample from the subject, wherein an increased amount of total cfDNA molecules from the sample as compared to a control amount correlates with the likelihood to benefit from the selected clinical intervention.

In some embodiments, the measuring is achieved by an assay selected from quantitative PCR (qPCR), digital droplet PCR (ddPCR), a flourometric DNA quantification assay and a spectroscopic DNA quantification assay.

4 In some embodiments, the biological sample is a blood sample, a serum sample, a plasma sample, a urine sample, a bronchoalveolar lavage sample, or a saliva sample.

In some embodiments, the subject is monitored by measuring the amount of total cfDNA molecules periodically.

In some embodiments, the increased severity and progression of SARS-CoV2 infection is reflected by admission to an intensive care unit and/or need for mechanical ventilation.

In some embodiments, the method further comprises providing a therapeutic regimen to the subject based on the result of the assessment.

In some embodiments, the selected clinical intervention comprises administration of one or more of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant. In some embodiments, the antiviral agent comprises remdesivir. In some embodiments, the steroid comprises dexamethasone.

In some embodiments, the clinical intervention comprises one or more of mechanical ventilation and oxygen supplementation.

Another aspect of the disclosure is directed to a method for assessing the severity and progression of a SARS-CoV2 infection in a subject comprising: obtaining cell-free DNA (cfDNA) molecules from a biological sample from the subject; measuring the amount of total cfDNA molecules in the biological sample, measuring the amount of tissue-specific cfDNA molecules in the biological sample, wherein the tissue-specific cfDNA molecules come from a tissue selected from the group consisting of erythroblast, kidney and liver; and determining the fraction of the amount of tissue-specific cfDNA molecules relative to the amount of total cfDNA molecules; wherein an increased fraction relative to a control is indicative of increased severity and disease progression in the subject.

In some embodiments, an increased fraction relative to a control is indicative of a high risk for mortality.

In some embodiments, the biological sample is a blood sample, a serum sample, a plasma sample, a urine sample, a bronchoalveolar lavage sample, or a saliva sample.

In some embodiments, the subject is monitored by measuring the amount of total and tissue-specific cfDNA molecules periodically.

In some embodiments, the method further comprises providing a therapeutic regimen to the subject based on the result of the assessment.

In some embodiments, the measuring of the amount of tissue-specific cfDNA comprises: determining the profiles of an epigenetic marker within the cfDNA molecules, wherein the epigenetic marker displays tissue-specific profiles; and identifying the tissues of origin of the cfDNA molecules based on the profiles determined.

In some embodiments, the epigenetic marker is selected from the group consisting of a DNA modification, a histone modification, and nucleosome positioning.

In some embodiments, the DNA modification is DNA methylation or DNA hydroxymethylation. In some embodiments, the histone modification is selected from the group consisting of acetylation, methylation, phosphorylation, ubiquitylation, GlcNAcylation, citrullination, krotonilation, and isomerization.

In some embodiments, the determining the profiles of the epigenetic marker comprises determining the sequences of the cfDNA molecules. In some embodiments, the profile of DNA methylation is determined by bisulfite treatment or enzymatic DNA methylation analysis. In some embodiments, the profile of DNA hydroxymethylation is determined by a pull down assay, a selective labeling assay, or an oxidative bisulfite sequencing assay. In some embodiments, the profile of histone modification is detected by a pull-down assay. In some embodiments, the nucleosome positioning is determined by a nucleosome positioning assay. In some embodiments, the determining the profiles of the epigenetic marker is achieved without determining the sequences of the cfDNA molecules. In some embodiments, the determining is achieved by a PCR assay selected from quantitiative PCR (qPCR) and digital droplet PCR (ddPCR).

In some embodiments, the assay comprises amplifying cfDNA molecules from regions of the genome that have specific epigenetic markers.

Another aspect of the disclosure is directed to a method for detecting tissue damage in a subject suffering from a SARS-CoV2 infection comprising: obtaining cfDNA molecules from a biological sample from the subject; determining the profiles of an epigenetic marker within the cfDNA molecules, wherein the epigenetic marker displays tissue-specific profiles; identifying the tissues of origin of the cfDNA molecules based on the profiles determined; and measuring the level of cfDNA molecules from an identified tissue of origin, wherein (i) the level or (ii) an increased level of cfDNA molecules from said identified tissue of origin as compared to a control level, is indicative of damage in said identified tissue of origin.

In some embodiments, the epigenetic marker is selected from the group consisting of a DNA modification, a histone modification, and nucleosome positioning. In some embodiments, the DNA modification is DNA methylation or DNA hydroxymethylation. In some embodiments, the histone modification is selected from the group consisting of acetylation, methylation, phosphorylation, ubiquitylation, GlcNAcylation, citrullination, krotonilation, and isomerization.

In some embodiments, the biological sample is a blood sample, a serum sample, a plasma sample, a urine sample, a bronchoalveolar lavage sample, or a saliva sample. In some embodiments, the tissues of origin comprise a solid organ. In some embodiments, the solid organ is an organ selected from kidney, liver, spleen, and pancreas. In some embodiments, the cfDNA molecules are from one or more organs selected from skin, heart, kidney, liver, lungs, stomach, bladder or pancreas.

Another aspect of the disclosure is directed to a method for treating a SARS-CoV2 infection in a subject, comprising: measuring the amount of total cell-free DNA (cfDNA) molecules in a biological sample from the subject; and treating the subject with a clinical intervention or adjusting the ongoing clinical intervention when an increased level of total cfDNA molecules from the sample as compared to a control level is measured.

In some embodiments, the selected clinical intervention comprises administration of one or more of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant.

In some embodiments, the antiviral comprises remdesivir.

In some embodiments, the steroid comprises dexamethasone.

In some embodiments, the clinical intervention comprises one or more of mechanical ventilation and oxygen supplementation.

In some embodiments, the measuring is achieved by an assay selected from quantitative PCR (qPCR), digital droplet PCR (ddPCR), a flourometric DNA quantification assay and a spectroscopic DNA quantification assay.

In some embodiments, the biological sample is a blood sample, a serum sample, a plasma sample, a urine sample, a bronchoalveolar lavage sample, or a saliva sample.

In some embodiments, the subject is monitored by measuring the amount of total cfDNA molecules periodically.

Another aspect of the disclosure is directed to a method for treating a SARS-CoV2 infection in a subject comprising: obtaining cell-free DNA (cfDNA) molecules from a biological sample from the subject; measuring the amount of total cfDNA molecules in the biological sample; measuring the amount of tissue-specific cfDNA molecules in the biological sample, wherein the tissue-specific cfDNA molecules come from a tissue selected from the group consisting of erythroblast, kidney and liver; determining the fraction of the amount of tissue-specific cfDNA molecules relative to the amount of total cfDNA molecules; and treating the subject with a clinical intervention or adjusting the ongoing clinical intervention when the fraction of the amount of tissue-specific cfDNA molecules is increased relative to a control.

In some embodiments, an increased fraction relative to a control is indicative of a high risk for mortality.

In some embodiments, the biological sample is a blood sample, a serum sample, a plasma sample, a urine sample, a bronchoalveolar lavage sample, or a saliva sample.

In some embodiments, the subject is monitored by measuring the amount of total and tissue-specific cfDNA molecules periodically.

In some embodiments, the measuring of the amount of tissue-specific cfDNA comprises: determining the profiles of an epigenetic marker within the cfDNA molecules, wherein the epigenetic marker displays tissue-specific profiles; and identifying the tissues of origin of the cfDNA molecules based on the profiles determined.

Another aspect of the disclosure is directed to a method of detecting a microbial co-infection of a subject suffering from a SARS-CoV2 infection comprising: obtaining cell-free DNA (cfDNA) molecules from a biological sample from the subject; determining the sequences of the cfDNA molecules; and identifying the presence of a cfDNA sequence of a microbial species other than SARS-CoV2, thereby detecting a co-infection by the microbial species.

In some embodiments, the extracted cfDNA molecules are bisulfite treated before determining the sequences of the cfDNA molecules.

In some embodiments, the method further comprises treating the subject with an anti-microbial agent when a microbial cfDNA sequence is identified in the biological sample.

In some embodiments, the anti-microbial agent is an anti-bacterial or anti-fungal agent.

In some embodiments, the anti-microbial agent is an anti-viral agent.

In some embodiments, the biological sample is a blood sample, a serum sample, a plasma sample, a urine sample, a bronchoalveolar lavage sample, or a saliva sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A - 1C. Study design. (A) Two independent cohorts were used in our study: First, a high frequency collection cohort with 5 SARS-CoV2 patients (n = 52 samples) and 6 SARS-CoV2 negative, RNA-virus positive patients (n = 6 samples). Second, a randomized control trial of 28 SARS-CoV2 patients with plasma at serial time points (n = 52 samples). 4 healthy individuals volunteered plasma for cell-free DNA analysis. (B) Experimental workflow. cfDNA is extracted from plasma and whole-genome bisulfite sequencing is performed. In parallel, methylation profiles of cell and tissue genomes are obtained from publicly-available databases. cfDNA methylation profiles are compared to those of cell and tissue references to infer relative contributions of tissues to the cfDNA mixtures. (C) UMAP of differentially methylated regions for isolated cell and tissue types used as a reference.

FIGS. 2A - 2E. High frequency collection cohort from UCSF. (A)-(B) Patient-specific relative tissue contributions for SARS-CoV2 patients (A) and other RNA-virus infection patients (B) Triangles indicate sampling times. (C) Heatmaps of Bray-Curtis dissimilarity. (D) Scatterplot of patient-specific Bray-Curtis dissimilarity (left) and boxplot of Bray-Curtis dissimilarity between cfDNA tissue proportions from samples collected from either the same day, the same person, or from the high-frequency collection cohort (right). (E) Comparison of tissue fraction of four cell and tissue types (neutrophil, erythroblast, lung and liver) between SARS-CoV2 positive patients and other RNA-virus positive patients. *: p-value < 0.05; **: p-value < 0.01; ***: p-value < 0.001 (p-values calculated using a Wilcoxon test)

FIGS. 3A-3G. Randomized control trial cohort from MUHC. (A) Patient sample-collection map by day of enrollment into the study. (B) Relative proportion of cfDNA derived from four cell and tissue types (neutrophil, erythroblast, lung, liver) by hospitalization status (p-values calculated using a Wilcoxon test). (C) Absolute cfDNA concentrations compared to the WHO ordinal scale for COVID progression. Blue shading indicates ordinal scores requiring admittance to the intensive care unit (ICU). (D) Receiver operating characteristic (ROC) analysis of the performance of absolute cfDNA concentration of different tissues (lung, erythroblast and total) in distinguishing patients presenting with ordinal scales from 4-6 (hospitalized) and 7-9 (hospitalized in the ICU). (E)-(G) Scatterplot comparisons between relative proportions of erythroblast cfDNA fraction and hemoglobin (E), liver cfDNA fraction and alanine aminotransferase (ALT) (F) and total cfDNA concentration and lactase dehydrogenase (LDH) (G). Green shading indicates normal levels. *: p-value < 0.05; **: p-value < 0.01; ***: p-value < 0.001.

### DETAILED DESCRIPTION

The inventors have developed novel methods for assessing the severity and progression of a SARS-CoV2 infection in a subject using cell-free DNA (cfDNA). In some embodiments, the methods of the instant disclosure provide independent prognostic and predictive parameters that can be used to determine how a SARS-CoV2 infection (a.k.a., COVID-19) will develop. In some embodiments, the methods of the instant disclosure determine the likelihood of a COVID-19 patient to benefit from a specific clinical intervention (e.g., a steroid treatment, an antiviral treatment, among others).

The inventors have investigated the utility of circulating cfDNA in a biological sample as an analyte i) to broadly monitor cell, tissue, and organ injury due to a SARS-CoV2 infection, ii) to assess disease severity and predict disease outcomes, and iii) to elucidate the multi-organ involvement that characterizes a SARS-CoV2 infection.

The inventors found that the total amount of circulating cfDNA accurately reflects a clinical status of a subject, with an increase in total cfDNA being strongly associated with admission to the intensive care unit and need for mechanical ventilation. Specifically, the inventors observed a strong correlation between the total cfDNA concentration isolated from a biological sample (e.g., plasma, serum, or whole blood or urine) and the WHO clinical progression scores.

The inventors also observed a striking increase, both in terms of proportion and total abundance (concentration), of tissue-specific cfDNA, e.g., cfDNA derived from different tissues such as red blood cell precursors (erythroblasts), liver or kidney, when compared to patients infected with other RNA viruses and healthy controls. The total burden of cfDNA correlated with the WHO ordinal scale for disease progression and can provide a marker of disease severity that is straightforward to adopt. Specifically, the inventors found that tissue-specific cfDNA (e.g., erythroblast cfDNA) proportions at any timepoint are highly predictive of in-hospital mortality.

The inventors have recognized the utility of cfDNA profiling as a diagnostic tool for the early detection and monitoring of cell and tissue injury associated with a SARS-CoV2 infection. A minimally invasive molecular blood test that can inform cell, tissue and organ specific injury due to a SARS-CoV2 infection has the potential to alleviate the impact of the COVID crisis i) by providing objectively quantifiable prognostic parameters and allowing a more granular assessment of clinical severity at the time of presentation; and ii) by providing a surrogate biomarker that can be included in clinical trials of candidate COVID-19 treatments. Total cfDNA, which can be extracted and measured within as few as 3 hours, can be used in the context of clinical trials and patient management to provide additional insights into the patient’s state.

### Methods for Assessing the Severity and Progression of a SARS-CoV2 Infection Using Amount of Total cfDNA

In one aspect, the disclosure is directed to a method for assessing the severity and progression of a SARS-CoV2 infection in a subject comprising measuring the amount of total cfDNA molecules in a biological sample from the subject, wherein an increased amount of total cfDNA molecules from the sample as compared to a control amount is indicative of increased severity and disease progression of SARS-CoV2 infection in the subject. In some embodiments, the control amount is measured from a healthy subject (a subject that does not have a SARS-CoV2 infection, a subject that is SARS-CoV2 negative in a nucleic acid-based test). In some embodiments, the control amount is measured from an earlier sample taken from the same subject, wherein the sample is taken before the subject has the SARS-CoV2 infection. In some embodiments, the control amount is a total cfDNA amount measurement taken from the same subject at an earlier time point of the SARS-CoV2 infection.

In some embodiments, a plurality of samples are taken from the subject over a period of time, and the trend of the amount of total cfDNA in the plurality of samples is used to assess the severity and progression of a SARS-CoV2 infection in the subject. As used herein, a “trend” means that at least three samples taken from the subject at different consecutive timepoints (samples taken periodically, e.g., every 3 hours, every 6 hours, every 9 hours, every 12 hours, every 18 hours, every 24 hours, every other day, every three days, every four days, every five days, every six days, every week, every ten days or every two weeks or with another regular interval) show the same direction of change. For instance, if at least three samples taken at different consecutive timepoints show increasing amounts of total cfDNA in time, this is an increasing trend. Similarly, if at least three samples taken at different consecutive timepoints show decreasing amounts of total cfDNA in time, this is an increasing trend.

In some embodiments, when a plurality of samples shows an increasing trend for the amount of total cfDNA, the subject is determined to have an increasing severity of SARS-CoV2 infection. In some embodiments, when a plurality of samples shows a decreasing trend for the amount of total cfDNA, the subject is determined to have a decreasing severity of SARS-CoV2 infection.

In some embodiments, the amount of total cfDNA molecules from a sample from a subject suffering from a SARS-CoV2 infection is at least 2 times, at least 3 times, at least 4 times, at least 5 times, at least 6 times, at least 7 times, at least 8 times, at least 9 times, at least 10 times, at least 15 times or more increased as compared to the control sample.

In another aspect, the disclosure is directed to a method for assessing the likelihood of benefit of a selected clinical intervention given to a subject suffering from a SARS-CoV2 infection comprising measuring the amount of total cfDNA molecules in a biological sample from the subject, wherein an increased amount of total cfDNA molecules from the sample as compared to a control amount correlates with the likelihood to benefit from the selected clinical intervention. In some embodiments, the control amount is a total cfDNA amount measurement taken from the same subject at an earlier time point of the SARS-CoV2 infection. In some embodiments, the selected clinical intervention comprises administration of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant, or a combination thereof. In some embodiments, the antiviral agent is selected from remdesivir, favipiravir or merimepodib. In some embodiments, the steroid comprises dexamethasone. In some embodiments, the clinical intervention comprises one or more of mechanical ventilation and oxygen supplementation.

In some embodiments, the amount of total cfDNA is measured by an assay selected from quantitative PCR (qPCR), digital droplet PCR (ddPCR), a fluorometric DNA quantification assay (e.g., the PicoGreen assay (commercially available, e.g., from ThermoFisher Scientific), a Qubit assay (commercially available, e.g., from ThermoFisher Scientific), or a hydroxymethylated DNA quantification assay), a spike-in assay, a spectroscopic DNA quantification assay (e.g., an assay that involves measuring absorbance at 260 nm/280 nm), gel electrophoresis and capillary electrophoresis assays, and assays based on fluorescence in-situ hybridization. An independent control can be included for batch effects.

In some embodiments, the biological sample is a blood sample, serum sample, plasma sample, urine sample, saliva sample, or bronchoalveolar lavage sample.

In some embodiments, the subject is monitored by measuring the amount of total cfDNA molecules periodically. As used herein, the term “periodically” refers to every 3 hours, every 4 hours, every 5 hours, every 6 hours, every 8 hours, every 12 hours, every day, every other day, every three days, every four days, every five days, every six days, every week, every ten days or every two weeks.

In some embodiments, the increased severity and progression of SARS-CoV2 infection is reflected by admission of the subject to an intensive care unit and/or need for mechanical ventilation.

In some embodiments, the subject is provided with a therapeutic regimen based on the assessment. In some embodiments, providing a therapeutic regiment includes providing a new therapy, or adjusting an ongoing therapy (e.g., adjusting dosage and/or schedule), or providing an alternative therapy to an ongoing therapy (e.g., when the ongoing therapy is found to be ineffective). In some embodiments, the therapeutic regimen comprises administration of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant, or a combination thereof. In some embodiments, the antiviral agent is selected from remdesivir, favipiravir or merimepodib. In some embodiments, the steroid comprises dexamethasone. In some embodiments, the clinical intervention comprises one or more of mechanical ventilation and oxygen supplementation.

### Methods for Assessing the Severity and Progression of a SARS-CoV2 Infection Using Tissue-Specific cfDNA Amounts

Another aspect of the disclosure is directed to a method for assessing the severity and progression of a SARS-CoV2 infection in a subject comprising obtaining cell-free DNA (cfDNA) molecules from a biological sample from the subject; measuring the amount of total cfDNA molecules in the biological sample, measuring the amount of tissue-specific cfDNA molecules in the biological sample, and determining the fraction of the amount of tissue-specific cfDNA molecules relative to the amount of total cfDNA molecules; wherein an increased fraction of tissue-specific cfDNA relative to a control is indicative of increased severity and disease progression in the subject. In some embodiments, the control tissue-specific cfDNA fraction (tissue-specific cfDNA/total cfDNA) is measured from a healthy subject (a subject that does not have a SARS-CoV2 infection, a subject that is SARS-CoV2 negative in a nucleic acid-based test). In some embodiments, the control tissue-specific cfDNA fraction is measured from an earlier sample taken from the same subject, wherein the sample is taken before the subject has the SARS-CoV2 infection. In some embodiments, the control amount is a tissue-specific cfDNA fraction measured from the same subject at an earlier time point of the SARS-CoV2 infection.

In some embodiments, an increased fraction of tissue-specific cfDNA relative to a control is indicative of a high risk for mortality.

In some embodiments, the tissue-specific cfDNA molecules come from a tissue selected from the group consisting of erythroblast (“erythroblast cfDNA”), kidney (“kidney cfDNA”), liver (“liver cfDNA”), lung (“lung cfDNA”), spleen (“spleen cfDNA”), pancreas (“pancreas cfDNA”), skin (“skin cfDNA”), heart (“heart cfDNA”), and bladder (“bladder cfDNA”). In some embodiments, the tissue of origin for the measured tissue-specific cfDNA is erythroblast and the increased fraction of erythroblast cfDNA is above 0.3, above 0.4, above 0.5, above 0.6 or higher. In some embodiments, the tissue of origin for the measured tissue-specific cfDNA is liver and the increased fraction of liver cfDNA is above 0.2, above 0.3, above 0.4, above 0.5, above 0.6 or higher. In some embodiments, the tissue of origin for the measured tissue-specific cfDNA is kidney and the increased fraction of kidney cfDNA is above 0.2, above 0.3, above 0.4, above 0.5, above 0.6 or higher. In some embodiments, the tissue of origin for the measured tissue-specific cfDNA is lung and the increased fraction of lung cfDNA is above 0.2, above 0.3, above 0.4, above 0.5, above 0.6 or higher. In some embodiments, the tissue of origin for the measured tissue-specific cfDNA is spleen and the increased fraction of spleen cfDNA is above 0.2, above 0.3, above 0.4, above 0.5, above 0.6 or higher. In some embodiments, the tissue of origin for the measured tissue-specific cfDNA is pancreas and the increased fraction of pancreas cfDNA is above 0.2, above 0.3, above 0.4, above 0.5, above 0.6 or higher. In some embodiments, the tissue of origin for the measured tissue-specific cfDNA is skin and the increased fraction of skin cfDNA is above 0.2, above 0.3, above 0.4, above 0.5, above 0.6 or higher. In some embodiments, the tissue of origin for the measured tissue-specific cfDNA is bladder and the increased fraction of bladder cfDNA is above 0.2, above 0.3, above 0.4, above 0.5, above 0.6 or higher. In some embodiments, the tissue of origin for the measured tissue-specific cfDNA is spleen and the increased fraction of spleen cfDNA is above 0.2, above 0.3, above 0.4, above 0.5, above 0.6 or higher.

In some embodiments, the sample comprises more than one type of cfDNA from a plurality (at least two different types) of tissues of origin.

In some embodiments, the biological sample is a blood sample, serum sample, plasma sample or urine sample.

In some embodiments, the subject is monitored by measuring the amount of total and tissue-specific cfDNA molecules periodically. In some embodiments, the subject is monitored by measuring the amount of total cfDNA molecules every 6 hours, every 12 hours, every day, every other day, every three days, every four days, every five days, every six days, every week, or every ten days.

In some embodiments, the increased severity and progression of SARS-CoV2 infection is reflected by admission to an intensive care unit and/or need for mechanical ventilation.

In some embodiments, the subject is provided with a therapeutic regimen based on the assessment. In some embodiments, providing a therapeutic regiment includes providing a new therapy, or adjusting an ongoing therapy (e.g., adjusting dosage and/or schedule), or providing an alternative therapy to an ongoing therapy (e.g., when the ongoing therapy is found to be ineffective).

In some embodiments, the therapeutic regimen comprises administration of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant, or a combination thereof. In some embodiments, the antiviral agent is selected from remdesivir, favipiravir or merimepodib. In some embodiments, the steroid comprises dexamethasone. In some embodiments, the clinical intervention comprises one or more of mechanical ventilation and oxygen supplementation.

In some embodiments, the measuring of the amount of tissue-specific cfDNA comprises determining the profiles of an epigenetic marker within the cfDNA molecules as described below (see the section entitled “Epigenetic Profiling” below), wherein the epigenetic marker displays tissue-specific profiles; and identifying the tissues of origin of the cfDNA molecules based on the profiles determined.

### Methods for Detecting SARS-CoV2 Infection-Related Tissue Damage

In one aspect, the disclosure is directed to a method for detecting tissue damage or tissue turnover (renewal of tissue due to cell death within the tissue) in a subject suffering from a SARS-CoV2 infection. In some embodiments, the method for detecting tissue damage in a subject suffering from a SARS-CoV2 infection comprises obtaining cfDNA molecules from a biological sample from the subject; determining the profiles of an epigenetic marker within the cfDNA molecules as described below (see the section entitled “Epigenetic Profiling” below), wherein the epigenetic marker displays tissue-specific profiles; identifying the tissues of origin of the cfDNA molecules based on the profiles determined; and measuring the level of cfDNA molecules from an identified tissue of origin, wherein (i) the level or (ii) an increased level of cfDNA molecules from said identified tissue of origin as compared to a control level, is indicative of damage or increased tissue turnover in said identified tissue of origin. In some embodiments, the level of cfDNA molecules is the absolute amount of cfDNA from a tissue (molecules per volume). In some embodiments, the level of cfDNA molecules is the relative amount of cfDNA from a tissue (as a % of total cfDNA). In some embodiments, the control tissue-specific cfDNA fraction (tissue-specific cfDNA/total cfDNA) is measured from a healthy subject (a subject that does not have a SARS-CoV2 infection, a subject that is SARS-CoV2 negative in a nucleic acid-based test). In some embodiments, the control tissue-specific cfDNA fraction is measured from an earlier sample taken from the same subject, wherein the sample is taken before the subject has the SARS-CoV2 infection. In some embodiments, the control amount is a tissue-specific cfDNA fraction measured from the same subject at an earlier time point of the SARS-CoV2 infection.

In some embodiments, the tissues of origin comprise a solid organ. In some embodiments, the solid organ is an organ selected from kidney, liver, spleen, and pancreas.

In some embodiments, the tissues of origin of the cfDNA molecules are from one or more organs selected from skin, heart, spleen, kidney, liver, lungs, stomach, bladder or pancreas.

### Methods of Treatment

In one aspect, the disclosure is directed to a method for treating a SARS-CoV2 infection in a subject comprising measuring the amount of total cfDNA molecules in a biological sample from the subject, and treating the subject with a clinical intervention or adjusting the ongoing clinical intervention when an increased level of total cfDNA molecules from the sample as compared to a control level is measured. In some embodiments, the control amount is measured from a healthy subject (a subject that does not have a SARS-CoV2 infection, a subject that is SARS-CoV2 negative in a nucleic acid-based test). In some embodiments, the control amount is measured from an earlier sample taken from the same subject, wherein the sample is taken before the subject has the SARS-CoV2 infection. In some embodiments, the control amount is a total cfDNA amount measurement taken from the same subject at an earlier time point of the SARS-CoV2 infection.

In some embodiments, the selected clinical intervention comprises administration of a steroid, an antiviral compound, a non-steroidal anti-inflammatory drug (NSAID), an ACE inhibitor, an angiotensin receptor blocker, convalescent plasma, an antibiotic, Interferon β, tocilizumab, and an anticoagulant, or a combination thereof. In some embodiments, the antiviral compound is selected from remdesivir, favipiravir or merimepodib. In some embodiments, the steroid comprises dexamethasone. In some embodiments, the clinical intervention comprises one or more of mechanical ventilation and oxygen supplementation.

In some embodiments, the amount of total cfDNA is measured by an assay selected from quantitative PCR (qPCR), digital droplet PCR (ddPCR), a flourometric DNA quantification assay (e.g., the PicoGreen assay, or a Hydroxymethylated DNA Quantification assay), a spike-in assay, and a spectroscopic DNA quantification assay (e.g., an assay that involves measuring absorbance at 260 nm/280 nm).

Another aspect of the disclosure is directed to a method for treating a SARS-CoV2 infection in a subject comprising obtaining cell-free DNA (cfDNA) molecules from a biological sample from the subject; measuring the amount of total cfDNA molecules in the biological sample; measuring the amount of tissue-specific cfDNA molecules in the biological sample; determining the fraction of the amount of tissue-specific cfDNA molecules relative to the amount of total cfDNA molecules; treating the subject with a clinical intervention or adjusting the ongoing clinical intervention when the fraction of the amount of tissue-specific cfDNA molecules is increased relative to a control.

In some embodiments, an increased fraction of tissue-specific cfDNA relative to a control is indicative of a high risk for mortality.

In some embodiments, the tissue-specific cfDNA molecules come from a tissue selected from the group consisting of erythroblast (“erythroblast cfDNA”), kidney (“kidney cfDNA”) and liver (“liver cfDNA”). In some embodiments, the tissue of origin for the measured tissue-specific cfDNA is erythroblast and the increased fraction of erythroblast cfDNA is above 0.3, above 0.4, above 0.5, above 0.6 or higher. In some embodiments, the tissue of origin for the measured tissue-specific cfDNA is liver and the increased fraction of liver cfDNA is above 0.2, above 0.3, above 0.4, above 0.5, above 0.6 or higher. In some embodiments, the tissue of origin for the measured tissue-specific cfDNA is kidney and the increased fraction of kidney cfDNA is above 0.2, above 0.3, above 0.4, above 0.5, above 0.6 or higher. In some embodiments, the tissue of origin for the measured tissue-specific cfDNA is lung and the increased fraction of lung cfDNA is above 0.2, above 0.3, above 0.4, above 0.5, above 0.6 or higher. In some embodiments, the tissue of origin for the measured tissue-specific cfDNA is spleen and the increased fraction of spleen cfDNA is above 0.2, above 0.3, above 0.4, above 0.5, above 0.6 or higher. In some embodiments, the tissue of origin for the measured tissue-specific cfDNA is pancreas and the increased fraction of pancreas cfDNA is above 0.2, above 0.3, above 0.4, above 0.5, above 0.6 or higher. In some embodiments, the tissue of origin for the measured tissue-specific cfDNA is skin and the increased fraction of skin cfDNA is above 0.2, above 0.3, above 0.4, above 0.5, above 0.6 or higher. In some embodiments, the tissue of origin for the measured tissue-specific cfDNA is bladder and the increased fraction of bladder cfDNA is above 0.2, above 0.3, above 0.4, above 0.5, above 0.6 or higher. In some embodiments, the tissue of origin for the measured tissue-specific cfDNA is spleen and the increased fraction of spleen cfDNA is above 0.2, above 0.3, above 0.4, above 0.5, above 0.6 or higher.

In some embodiments, the measuring of the amount of tissue-specific cfDNA comprises determining the profiles of an epigenetic marker within the cfDNA molecules as described below (see the section entitled “Epigenetic Profiling” below), wherein the epigenetic marker displays tissue-specific profiles; and identifying the tissues of origin of the cfDNA molecules based on the profiles determined.

In some embodiments, the biological sample is a blood sample, serum sample, plasma sample or urine sample.

In some embodiments, the subject is monitored by measuring the amount of total cfDNA molecules periodically. As used herein, the term “periodically” refers to every 6 hours, every 12 hours, every day, every other day, every three days, every four days, every five days, every six days, every week, every ten days or every two weeks.

In some embodiments, the increased severity and progression of SARS-CoV2 infection is reflected by admission to an intensive care unit and/or need for mechanical ventilation.

In some embodiments, the subject is provided with a therapeutic regimen based on the assessment. In some embodiments, providing a therapeutic regiment includes providing a new therapy, or adjusting an ongoing therapy (e.g., adjusting dosage and/or schedule), or providing an alternative therapy to an ongoing therapy (e.g., when the ongoing therapy is found to be ineffective). In some embodiments, the therapeutic regimen comprises a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant, or a combination thereof. In some embodiments, the antiviral comprises remdesivir. In some embodiments, the steroid comprises dexamethasone. In some embodiments, the clinical intervention comprises one or more of mechanical ventilation and oxygen supplementation.

### Methods for Detecting Microbial Infections

Another aspect of this disclosure is directed to a method for detecting a microbial co-infection of a subject suffering from a SARS-CoV2 infection comprising: obtaining cell-free DNA (cfDNA) molecules from the biological sample; determining the sequences of the cfDNA molecules; and identifying the presence of a cfDNA sequence of a microbial species other than SARS-CoV2, thereby detecting a co-infection by the microbial species. Microbial cfDNAs can have a bacterial, fungal, or viral origin.

In some embodiments, the extracted cfDNA molecules are bisulfite treated before determining the sequences of the cfDNA molecules.

In some embodiments, the method comprises treating the subject with an anti-microbial agent when a microbial cfDNA sequence is identified in the biological sample. In some embodiments, the anti-microbial agent is an anti-bacterial or anti-fungal agent. In some embodiments, the anti-microbial agent is an anti-viral agent.

In some embodiments, the biological sample is a blood sample, a plasma sample, a serum sample or a urine sample.

### Epigenetic Profiling Workflow

In some embodiments, the methods of the disclosure include determining the profile of an epigenetic marker. In some embodiments, the epigenetic marker is selected from the group consisting of a DNA modification, a histone modification, and nucleosome positioning. In some embodiments, the DNA modification is DNA methylation or DNA hydroxymethylation. In some embodiments, the histone modification is selected from the group consisting of acetylation, methylation, phosphorylation, ubiquitylation, GlcNAcylation, citrullination, krotonilation, and isomerization.

In some embodiments, the determining the profiles of the epigenetic marker comprises determining the sequences of the cfDNA molecules.

In some embodiments, the profile of DNA methylation is determined by bisulfite treatment or enzymatic DNA methylation analysis. In some embodiments, the workflow of determining DNA methylation profiling comprises the following steps: cell-free DNA extraction, bisulfite treatment, library preparation, sequencing, and analysis. In some embodiments, the workflow further comprises plasma extraction from blood.

In some embodiments, an alternative to bisulfite treatment is used to detect cfDNA methylation.

In some embodiments, DNA methylation is detected using enzymatic DNA methylation analysis. In some embodiments, enzymatic DNA methylation analysis comprises using restriction enzymes that are methylation sensitive. In a specific embodiment, enzymatic DNA methylation analysis comprises using the enzyme HpaI. In some embodiments, the Enzymatic Methyl-seq Kit from NEB is used to detect cfDNA methylation. The Enzymatic Methyl-seq technique is used to uncover the same information as bisulfite treatment, but uses enzymes instead of chemicals. The other parts of the workflow are the same.

In some embodiments, the epigenetic profiling comprises alternatives to methylation markers. In some embodiments, other epigenetic marks that are tissue specific and maintained in cell-free DNA are used in the methods of this disclosure.

In some embodiments, the epigenetic profiling comprises DNA hydroxymethylation profiling. Hydroxymethylation is a chemical modification present on cytosines that is thought to be indicative of a gene being activated. Papers have described this chemical modification as tissue-specific. See, e.g., Song, Chun-Xiao, et al., Cell Research, 27.10 (2017): 1231-1242; Nestor, Colm E., et al., Genome research; 22.3 (2012): 467-477, incorporated herein in their entirety.

In some embodiments, determining hydroxymethylation profile of cfDNA is achieved by a pull down assay. In some embodiments, the pull-down assay utilizes engineered antibodies specific for hydroxymethylated cytosines can be used to capture hydroxymethylated-rich cell-free DNA.

In some embodiments, determining hydroxymethylation profile of cfDNA is achieved by a selective labeling assay (selective chemical labelling of hydroxymethylated cytosines). In some embodiments, selective labeling is achieved by a B-glucosyltransferase enzyme that adds a biotin group to the hydroxymethylated cytosines. Streptavidin beads can then be ligated to the biotin groups to pull out hydroxymethylated-rich cfDNA. The pulled down cfDNA is then sequenced

In some embodiments, determining hydroxymethylation profile of cfDNA is achieved by oxidative bisulfite sequencing. In some embodiments, oxidative bisulfite sequencing comprises a) splitting cfDNA into two groups;b) bisulfite-treating one half of the cfDNA sample, revealing which cytosines were methylated or hydroxymethylated; c) oxidizing the other half (which removes the hydroxymethyl group), and then bisulfite treating the oxidized half. This reveals which cytosines were methylated; and d) sequencing the split samples reveals which cytosines were methylated, and which were hydroxymethylated. These methods provide a single-base pair resolution on both hydroxymethylated and methylated sites.

In some embodiments, the epigenetic profiling comprises histone modifications. DNA in the genome is wrapped around nucleosomes. Nucleosomes are composed of 8 histones. Histone modifications are associated with gene expression and their presence can be detected to estimate the tissues of origin of cell free DNA. When DNA is out of the cell (through apoptosis, for example), it gets degraded. It is thought that DNA that is wrapped around histones, however, is more protected from degradation, and can therefore be captured and sequenced (most cfDNA we sequenced is histone-wrapped). The modifications on these histones are indicative of tissues of origin. See, Sadeh, Ronen, et al., bioRxiv (2019): 638643, incorporated herein in its entirety.

In some embodiments, probing histone modifications in cell free DNA is achieved by:
1- Performing a pull-down assay using antibodies specific for a histone modification. In some embodiments, the antibodies are specific for histone methylation, acetylation, phosphorylation, ubiquitylation, GlcNAcylation, citrullination, krotonilation, or isomerization. In some embodiments, the histone methylation-specific antibodies comprise antibodies against H3K4Me1, H3K4Me2, H3K4Me3, or H3K36Me3 modifications.
2- Sequencing pulled-down cfDNA.

In some embodiments, probing histone modifications in cell free DNA is achieved by nucleosome positioning. DNA that is wrapped in a nucleosome cannot be transformed into RNA. It needs to be unwrapped by enzymes to be transcribed. Therefore, when a cell dies and its genome is released, the areas that were being transcribed are degraded (because they are not transcribed). The cfDNA that are sequenced were not being transcribed. In theory, the areas of the genome that were not seen can be assumed to be actively transcribed. These patterns have been shown to be tissue-specific. See, Snyder, Matthew W., et al., Cell, 164.1-2 (2016): 57-68.; Sun, Kun, et al., Genome Research, 29.3 (2019): 418-427., incorporated in their entirety.

In some embodiments, probing nucleosome positioning is achieved by sequencing cell-free DNA (without bisulfite treatment).

### Determining Epigenetic Profiles Without Sequencing

In some embodiments, epigenetic profiling can be determined without sequencing the cfDNA. In some embodiments, epigenetic profiling is determined by performing quantitiative PCR (qPCR) or digital droplet PCR (ddPCR) (as described in Shemer, R. et al. Current Protocols in Molecular Biology, 127.1 (2019): e90; and Zemmour, Hai, et al., Nature Communications, 9.1 (2018): 1-9, both incorporated herein in their entirety).

In some embodiments, epigenetic profiling involves isolating cell-free DNA from a sample; amplifying cfDNA from regions of the genome that have specific epigenetic markers; detecting modified or unmodified epigenetic marks at a tissue-specific region using probes that can distinguish modified and unmodified epigenetic marks; and using a either a qPCR or ddPCR assay to detect a readout. In some embodiments, the primers and/or probes comprise fluorescent labels. In some embodiments, the fluorescent signal from the probe is measured as the readout and tissue composition of the cfDNA is inferred from the readout.

In some embodiments, the epigenetic profile is a DNA methylation profile. In some embodiments, determining the methylation profile does not comprise determining the sequence of the cfDNA.

In some embodiments, after bisulfite treatment, qPCR or ddPCR are used to amplify regions that comprise specific methylation markers that are tissue specific. The degree of amplification can be measured to estimate tissue-specific contributions to cell-free DNA.

In some embodiments, determining methylation profiles comprises: isolating cell-free DNA from a sample; amplifying cfDNA from regions of the genome that have methylation-specific markers; detecting methylation at a tissue-specific region; and using a either a qPCR or ddPCR assay to readout the fluorescent signal, and use the fluorescent signal to infer tissue composition of cfDNA. In some embodiments, determining methylation at a tissue-specific region is achieved by using probes that bind to either methylated or unmethylated cytosines.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

High frequency sampling. Clinical samples from UCSF were processed through protocols approved by the UCSF Institutional Review Board (protocol number 10-00476, 18-25287). Residual plasma was collected as part of routine clinical testing and stored at 4° C. for up to 5 days and subsequently stored at -80° C. until batched extraction. Plasma was initially isolated from blood by the clinical laboratory after centrifugation at approximately 800 g for 10 minutes. After storage, the plasma was centrifuged at 16,000 g for 10 minutes. CfDNA extraction was performed according to manufacturer recommendations (Qiagen Minlute Circulating Nucleic Acid Kit, reference #55204 or Qiagen EZ1 Virus Mini Kit v2.0 955134) at 0.4-1 mL plasma input.

Randomized clinical trial. Individuals diagnosed with COVID-19 were recruited to a randomized, controlled clinical trial at the McGill University Health Center, where they received either Lopinavir/ritonavir, or standard-of-care (Clinical Trial No: NCT04330690). Blood samples were collected under MUHC Research Ethics Board protocol 10-256 through standard venipuncture in standard blood collection tubes and immediately centrifuged at 850 g for 10 minutes. The supernatant was then transferred to new tubes, and centrifuged at 16,000 g for 10 minutes. Plasma-containing supernatant was collected and stored in DNA cryostorage vials (Eppendorf, reference #0030079400) at -80° C. Plasma was shipped overnight on dry ice from the McGill University Health Center (Montreal, Canada) to Cornell University (Ithaca, United-States). Plasma was stored at -80° C. until used for cfDNA extraction. cfDNA extraction was performed according to manufacturer recommendations (Qiagen Circulating Nucleic Acid Kit, reference #55114).

Healthy controls. Volunteers were recruited for blood donations through a protocol approved by the Cornell Institutional Review Board (protocol number 1910009101). Blood was collected in K2 EDTA tubes (BD, reference #366643) and immediately centrifuged at 1600 g for 10 minutes. The supernatant was transferred to new tubes, and centrifuged at 16,000 g for 10 minutes. Supernatant is then stored in DNA cryostorage vials (Thermo Scientific #363401) at -80° C. until cfDNA extraction. cfDNA extraction was performed according to manufacturer recommendations (Qiagen Circulating Nucleic Acid Kit, reference #55114).

Whole genome bisulfite sequencing. Bisulfite treatment of DNA converts cytosine residues to uracil but leaves methylated cytosines unaffected. DNA sequencing of bisulfite-treated cfDNA can be used to reveal methylation patterns with single nucleotide resolution. Because these patterns are cell, tissue and organ types specific, they can inform the origins of cfDNA. Following treatment with bisulfite, whole-genome sequencing (WGS) libraries were prepared according to manufacturer’s protocols (Zymo EZ Methylation-Gold kit, #D5005 and Swift Biosciences Accel-NGS Methyl-Seq DNA Library Kit #30024) using a dual indexing barcode strategy (Swift biosciences #38096, NEBNext Multiplex Oligos for Illumina E7500L, or custom primers). Paired-end DNA sequencing was performed on the Illumina NextSeq 500 (2x75bp) at Cornell University or the Illumina NovaSeq (2x150bp) at University of California San Francisco. Resulting paired-end fastq files were trimmed to 75bp for downstream analysis.

Human genome alignment. Adapter sequences were trimmed using BBDUK (BBTools software suite). Resulting sequences were aligned to the human genome (version hg19) and deduplicated using Bismark. Alignment files were filtered with a minimum mapping quality of 10 using SAMtools.

Reference methylomes and tissues of origin. Reference methylation profiles were obtained from publicly available datasets and international epigenetic consortium projects and processed as previously described. Briefly, files were downloaded and normalized to a standard 4 column BED format at single nucleotide resolution using hg19 coordinates. Differentially methylated regions (DMRs) were found using Metilene. Methylation densities within these DMRs were averaged. Tissues with methylation profiles highly dissimilar from the same tissues were removed. cfDNA methylation densities were extracted using Bismark and averaged over the DMRs. Tissues of origin were deconvoluted using a non-negative least squares approach.

cfDNA concentration measurement - MUHC patients. Plasma samples were processed in batches of 4 to 10 alongside a control containing 8 µL of approximately 150 ng/µL of synthetic oligos. DNA concentration measurements were performed after cfDNA extraction (Qubit Fluorometer 3.0) and the normalized concentration was calculated by multiplying the sample’s concentration by the input/output ratio of the control.

Depth of coverage. The depth of DNA sequencing coverage was calculated by dividing the number of mapped nucleotides to the autosomal chromosomes to the size of the non-N hg19 autosomal genome.

Bisulfite conversion efficiency. The bisulfite conversion efficiency achieved in experiments was estimated using MethPipe by calculating the reported methylation density of cytosines present at C[A/T/G] dinucleotides, which are rarely methylated in mammalian genomes.

Quality control filtering. Samples from the high frequency sampling cohort were selected for analysis if 10 or more spike-in molecules were identified after sequencing and were also filtered for sufficient depth of sequencing (>0.2x human genome). Samples from the randomized control trial cohort were sequenced to a minimum depth of 0.7x human genome coverage. All samples had a minimum bisulfite conversion efficiency of 96%.

Statistical analysis. All statistical analyses were performed in R, version 3.5.0. Groups were compared using the two-sided, nonparametric Wilcoxon test. If the data distributions were zero-skewed, a two-sided, 2-sample proportions test without continuity correction was performed.

Data availability. Genomic data will be hosted on the Sequence Read Archine. The code used to generate figures and analyze primary data is available at GitHub cfDNAme website.

### Example 2: Temporal Dynamics of Cell-free DNA Tissues-of-origin in Plasma of COVID-19 Patients

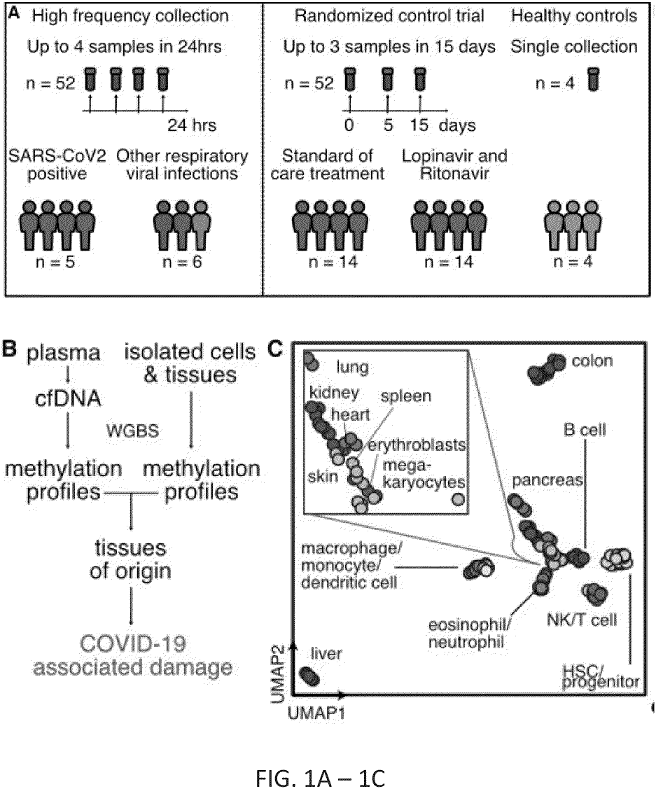

The inventors tested the utility of cfDNA to quantify cell, tissue and organ specific injury associated with COVID-19 in two independent patient cohorts from two different hospitals in North America (FIG. 1A). The inventors assayed a total of 104 plasma samples from 33 patients across these cohorts. The inventors performed shotgun DNA sequencing after bisulfite treatment to determine the tissues-of-origin of cfDNA isolated from plasma by methylation profiling. The inventors obtained 62 ± 35 million (mean ± standard deviation) paired-end reads per sample, leading to a per-base genome coverage of 1.3 ± 0.8. The inventors verified that we achieved a high bisulfite conversion efficiency for all samples (0.996 ± 0.005, Methods). To determine the cell, tissue and organ types that contribute cfDNA to the mixture in blood (Methods), we analyzed plasma cfDNA methylation profiles against a reference set of 147 cell, tissue and organ types using previously described bioinformatic approaches

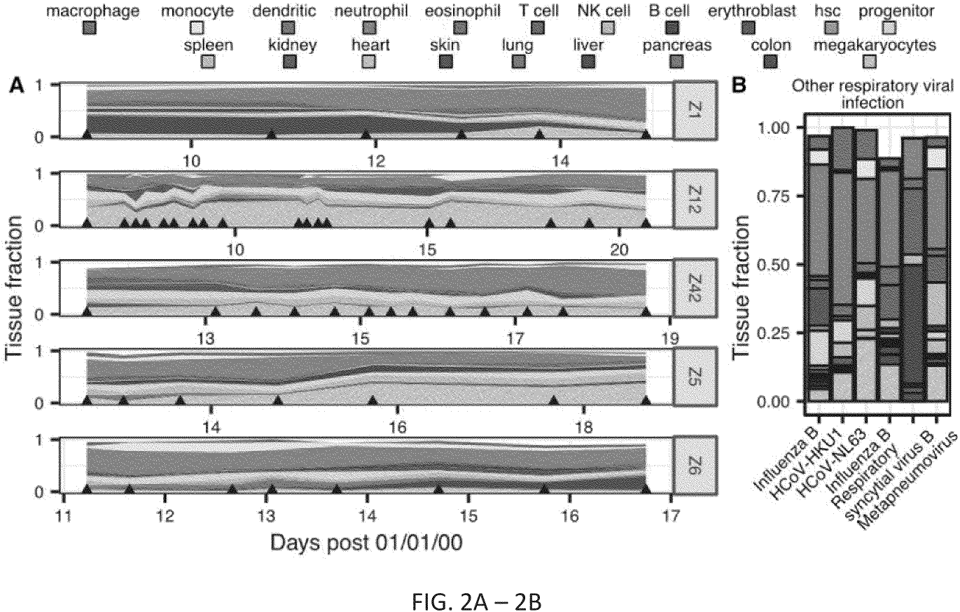

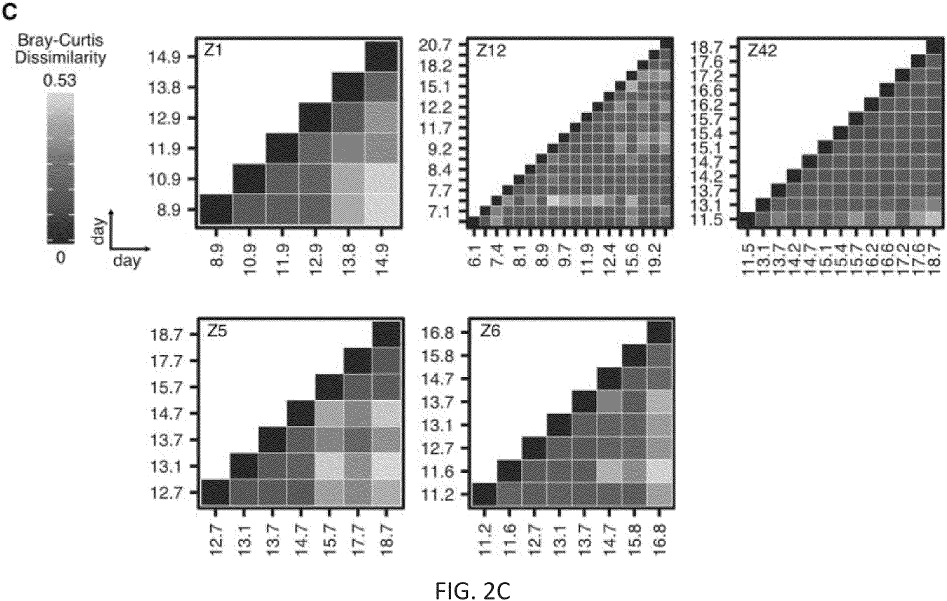

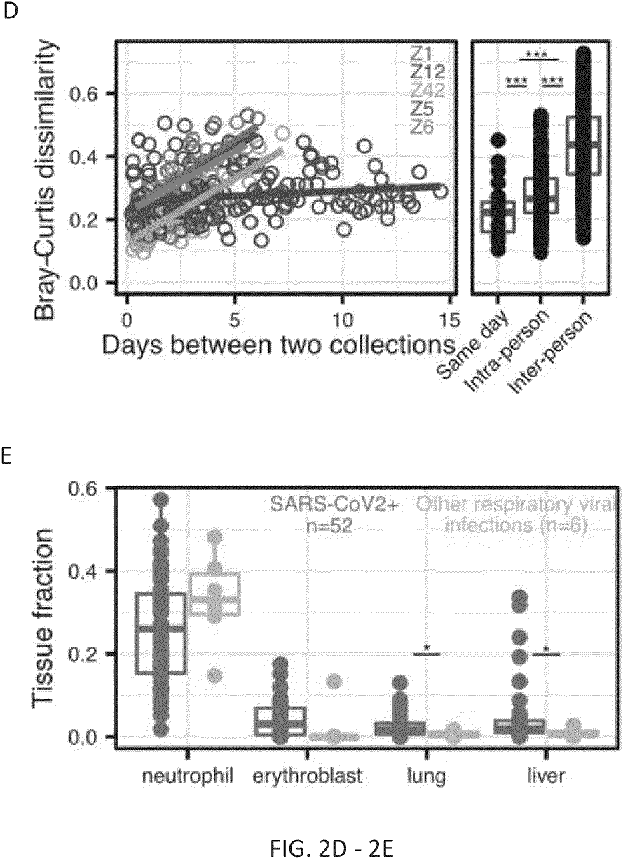

The inventors first assayed 52 serial samples collected at short time intervals from five patients with COVID-19 that were treated at University of California, San Francisco (UCSF) Medical Center (median of 8 samples per patient [range 6-18]). These plasma samples were residual from clinical testing and were collected from this group of patients over a treatment time-period of up to 14 days with up to four samples collected within 24 hours (median time between consecutive collections of 13 hours [range 5-64]). These samples allowed the inventors to study dynamic changes in cfDNA profiles in patients diagnosed with and treated for COVID-19 (FIG. 2A). Treatments included standard of care (n=2), remdesivir (n=1), hydroxychloroquine (n=1), or a combination of remdesivir, hydroxychloroquine, azithromycin and tocilizumab (n=1). In addition to plasma from COVID-19 patients, we performed cfDNA tissues-of-origin profiling for six samples collected from patients with other respiratory viral infection treated at the same hospital, including influenza B (n=2), Metapneumovirus (n=1), Coronavirus HKU1 (n=1), Coronavirus NL63 (n=1) and Respiratory syncytial virus B (n=1) (FIG. 2B). The inventors plotted the relative abundance of cfDNA derived from different cell, tissue, and organ types and found that differences in cfDNA profiles between individuals were larger than differences within individuals over the sampling period. For subjects Z1, Z5, Z6, and Z42 but not Z12, gradual changes were observed in the tissues-of-origin profiles over sampling periods of six to seven days. The inventors used the Bray Curtis dissimilarity to quantify the inter and intra-individual differences in cfDNA profiles (FIGS. 2C-D). This analysis confirmed the visual appearance of the tissues-of-origin profiles in FIG. 2A and demonstrated that the largest differences in cfDNA were found for samples collected from different individuals. Within subjects, smaller differences were observed for samples collected on the same day (FIG. 2D). Last, the Bray Curtis dissimilarity increased with time interval between samples for patients Z1, Z5, Z6, and Z42 but not for Z12. Together these analyses indicate that cfDNA profiles are subject specific, and that changes in cfDNA tissues-or-origin profiles occur gradually over days and not hours, therefore adequate longitudinal data can be collected every few days.

The inventors next compared the cfDNA tissues-of-origin profiles associated with COVID-19 versus those associated with respiratory infection with other viruses (FIG. 2E). The inventors found significant increases in the relative proportion of lung specific cfDNA in the blood of COVID-19 patients, which was likely related to COVID-19 associated tissue injury (2.5% vs 0.6%, p-value = 0.019, Wilcoxon). The inventors found a similar association with liver-derived cfDNA, potentially due to drug treatment related toxicity (5.0% vs 0.9%, p-value = 0.025, Wilcoxon). Strikingly, the inventors also observed an increase in the relative proportion of cfDNA derived from erythroblasts in the blood of COVID-19 patients compared to the control group (75% vs 17% samples with an erythroblast fraction greater than 0, p-value = 0.003, 2-sample proportions test, FIG. 2E). Erythroblasts are nucleated cells in the bone marrow from which red blood cells develop. The increase in cfDNA derived from red blood progenitor cells seen here may be an indirect consequence of the hypoxemia and/or cytokine-mediated anemia that characterize severe COVID-19, or may indicate a more direct involvement of coronavirus with red blood cell precursors. The inventors note that erythroblast cfDNA was elevated in a single patient in the control group. This patient was diagnosed with recurrent stage IV diffuse large B-cell lymphoma (FIGS. 2B and 2E).

### Example 3: Randomized Clinical Trial Cohort

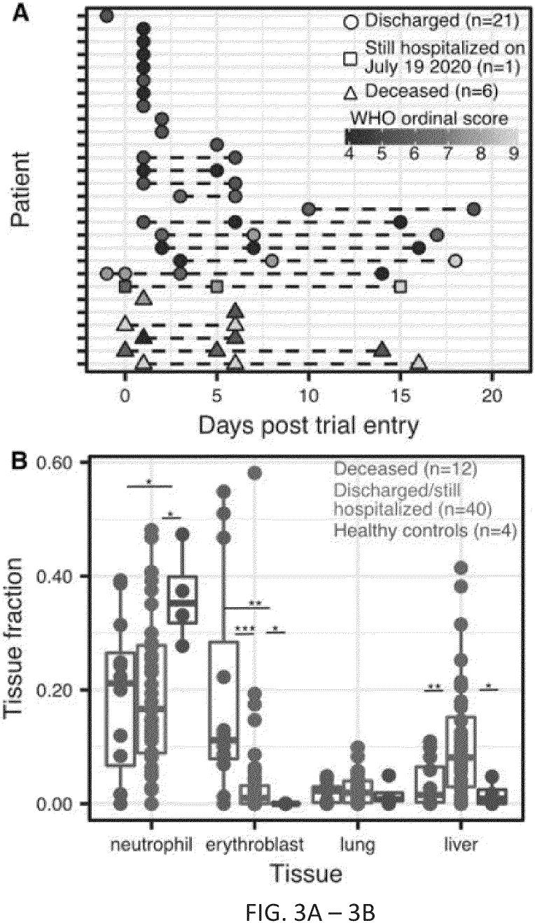

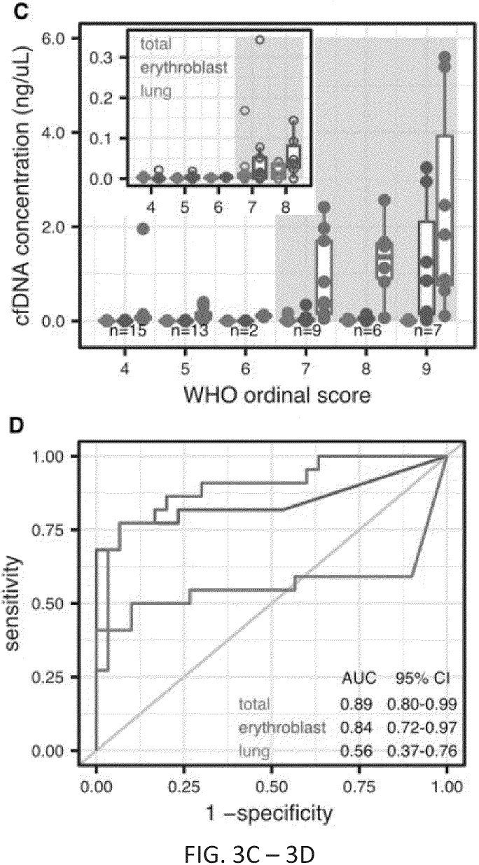

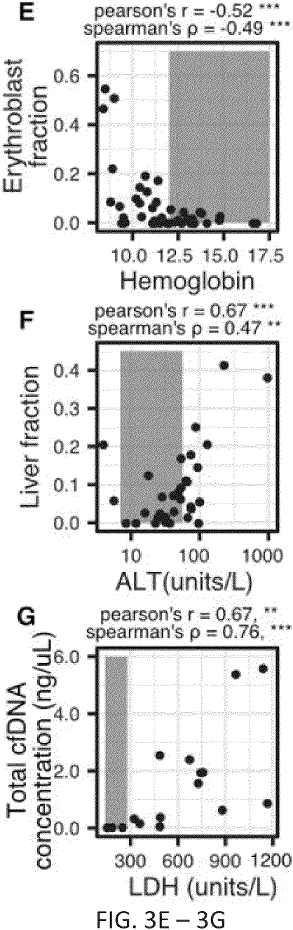

To test the robustness of these initial observations, the inventors assayed an additional 52 samples collected from 28 patients that were recruited into a randomized control trial at the McGill University Health Centre. Patients were assigned to either an experimental antiviral therapy consisting of a combination of Lopinavir and Ritonavir (brand name Kaletra) or to the standard of care. Of these patients, 14 were treated with the Lopinavir/Ritonavir, and 14 were treated with the standard of care. Of the 28 patients, 21 were discharged after treatment, one patient remains hospitalized as of July 19^{th}, 2020, and six patients died. Serial samples were collected from these patients at three predetermined time points: days 1, 5, and 15 after enrollment in the clinical trial, provided they remained hospitalized on the days of collection (FIG. 3A). The inventors determined the relative abundance of tissue-specific cfDNA using the approaches described above. In addition, the inventors quantified the absolute concentration of tissue-specific cfDNA by multiplying the proportion of tissue-specific cfDNA with the concentration of total cfDNA.

The inventors first compared the cfDNA tissues-of-origin profiles measured for these patients with the tissues-of-origin profiles for four healthy subjects (FIG. 3B). We found that 62% of samples from patients with COVID-19 had a higher concentration of lung cfDNA than the highest concentration measured for a healthy individual (p-value = 0.017, 2-sample proportions test). In addition, hospitalized patients with COVID-19 had both an elevated relative and absolute burden of cfDNA derived from the liver (liver fraction 9.1 vs 1.6%, p-value = 0.054, and 0.051 ng/µL vs 0.00029 ng/µL, p-value = 0.010, Wilcoxon). In addition to these tissue-specific features, the inventors again observed a significant increase in cfDNA derived from erythroblast cells for COVID-19 patients compared to healthy controls (7.7% vs 0%, p-value = 0.027, Wilcoxon; 65% vs 0% of samples showing erythroblast fraction greater than 0, p-value = 0.0099, 2-sample proportions test, FIG. 3B).

The inventors next compared cfDNA signatures for COVID-19 patients as function of disease severity, and found that erythroblast cfDNA proportions at any timepoint are predictive of in-hospital mortality (19.6% vs 4.1%, p-value = 0.0004, Wilcoxon). Receiver operating characteristic (ROC) analysis of the performance of the relative proportion of Erythroblast derived DNA to predict COVID-19 mortality yielded an area under the curve (AUC) of 0.83 (95% CI 0.69-0.98, [deceased n = 12; hospitalized or discharged n = 40]). Additionally, the instant analysis revealed that kidney cfDNA was significantly elevated in deceased COVID-19 patients (1.8% vs 0.5% vs 0.005% between deceased, non-deceased and healthy controls, p-value = 0.0018 between deceased and non-deceased COVID-19 patients).

The inventors then compared the cfDNA tissues-of-origin profiles to the WHO clinical progression scale for COVID-19 (FIG. 3C). The inventors found a strong association between the total cfDNA concentrations isolated from plasma and the WHO clinical progression scores (FIGS. 3C & 3D). Notably, a clinical score of 7 or greater (need for admission to the intensive care unit and mechanical ventilation), was associated with a sharp increase in the total burden of cfDNA (FIGS. 3C & 3D, mean 1.5 ng/µL vs 0.16 ng/µL, between clinical scores from 7 to 9 and 4 to 6, respectively; p-value = 1.5×10⁻ ⁶, Wilcoxon). ROC analysis of cfDNA concentrations to predict ordinal scores revealed AUCs of 0.89 (95% CI 0.80-0.99), 0.84 (95% CI 0.72-0.97) and 0.56 (95% CI 0.37-0.76) for total, erythroblast and lung cfDNA, respectively. Furthermore, samples taken from patients with a clinical score of 9 (use of extracorporeal membrane oxygenation [ECMO]) had significantly higher erythroblast-derived cfDNA than patients with a clinical score of 7-8 (1.23 ng/µL vs 0.06 ng/µL, p-value = 0.006, Wilcoxon). Patients on ECMO tend to bleed and require additional blood volumes, which may contribute to the increased erythroblast signal. However, erythroblast-derived cfDNA was significantly increased in patients with a clinical score of 7 or higher as well (FIGS. 3C & 3D, mean 0.43 ng/µL vs 0.003 ng/µL, p-value = 1.83×10⁻⁵, Wilcoxon).

Erythroblast and liver cfDNA contributions correlated with clinical metrics for anemia and liver damage, respectively (FIGS. 3E-3G). The inventors observed significant negative correlations between the proportion of erythroblast cfDNA and hematocrit and hemoglobin (Pearson’s R (R) = -0.51, Spearman’s ρ (ρ) = -0.37 and R = -0.52, ρ = -0.49, respectively). Similarly, positive correlations were observed between the proportion of liver-derived cfDNA and alanine aminotransferase (ALT) and aspartate transaminase (R = 0.63, ρ = 0.47 and R = 0.76, ρ = 0.24, respectively). The inventors did not observe a correlation between kidney-derived cfDNA and serum creatinine (R = 0.05, ρ = 0.09). The inventors found similar results when comparing the tissue-derived cfDNA concentration to these clinical markers (erythroblast cfDNA concentration vs hematocrit and hemoglobin: R = -0.42, ρ = -0.32 and R = -0.38, ρ = -0.45, respectively. Liver cfDNA concentration vs ALT and AST: R = 0.84, ρ = 0.52 and R = 0.20, ρ = 0.23, respectively. Kidney cfDNA concentration vs creatinine: R = 0.56, ρ = 0.20).

Recently lactate dehydrogenase (LDH) was identified as a strong predictor of COVID-19 outcome. LDH is found in virtually all cells and is a commonly used biomarker for tissue damage and hemolysis. The inventors found significant correlation between LDH and the proportion of erythroblast-derived cfDNA (R = 0.64, ρ = 0.65), and between LDH and total cfDNA (R = 0.67, ρ = 0.76). Together, these data suggest that cfDNA tissues-of-origin can be applied to resolve the specific tissues contributing to nonspecific detection of LDH in blood.

Finally, the inventors found no differences between in lung, liver, kidney or erythroblast-derived cfDNA for patients receiving standard of care, or the experimental lopinavir/ritonavir treatment. These data are in line with the results of recent clinical trials that treatment with lopinavir/ritonavir is not significantly different from standard of care treatment for COVID-19.

### Example 4

The inventors find significant support for the utility of cfDNA profiling as a diagnostic tool for the early detection and monitoring of cell and tissue injury associated with COVID-19. A minimally invasive molecular blood test that can inform cell, tissue and organ specific injury due to COVID-19 has the potential to alleviate the impact of the COVID crisis i) by providing quantifiable prognostic parameters and a more granular assessment of clinical severity at the time of presentation; and ii) by providing a surrogate biomarker that can be included in clinical trials of candidate COVID-19 treatments.

In line with the diverse clinical manifestations of COVID-19, the inventors find evidence for lung, liver and kidney injury in hospitalized patients with COVID-19. While lung-derived cfDNA was elevated in COVID-19 patients, the inventors did not find it to be a major contributor to plasma cfDNA. The level of lung specific cfDNA in plasma was similar to the levels observed in lung transplant patients that suffer acute lung transplant rejection and lung cancer patients. The inventors observed a striking correlation between the total abundance of circulating cfDNA in plasma and the WHO ordinal scale for disease progression. The inventors propose that the total abundance of cfDNA, which can be measured within one hour at a low cost, can be used in the context of clinical trials and patient management in the near term.

In addition to the practical application of cfDNA profiling to patient monitoring and COVID-19 risk stratification, the cfDNA methylation assay and data reported may help elucidate aspects of COVID-19 pathogenesis. The most significant cfDNA signature observed in the two cohorts relative to controls was an increase in cfDNA derived from erythroid or red blood progenitor cells. Given that cfDNA is estimated to have a half-life of about 1 hour and that the proportion of the erythroid lineage was relatively stable over several days, the elevated erythroid cfDNA is likely due to a continuous increased erythroid turnover. In support of elevated erythroid turnover and production, red blood cell distribution width (RDW), a measure of the variation in size of red blood cells (RBCs), was identified as an important prognostic predictor for severe COVID-19. The increased RDW was speculated to be associated with increased turnover of RBCs since increased reticulocytes or newly formed RBCs have a wider diameter.

However, the instant analysis demonstrated that there was no association with RDW and patient outcomes (mean 15.4 vs 14.0 between deceased and discharged or hospitalized, p-value = 0.2, Wilcoxon) and that erythroblast cfDNA was not strongly correlated with RDW (R = 0.26, ρ = 0.13 [with data from UCSF and MUCH]).

Increased erythroid turnover is likely due to erythroid destruction as the primary driver, followed by compensatory production, and is supported by anemia (Hgb < 13.5 g/dL for men and Hgb < 12 for women) found in 26 of 33 COVID-19 patients across both studies. Possible mechanisms include: i) excessive inflammation and cytokine storm, ii) hemophagocytosis in relation to inflammation, and iii) consumption in microthrombi. The inventors note that 18 of 33 patients in all studies, C-reactive protein (CRP) was elevated (> 10 mg/L). It is notable however that megakaryocytes proportions were not increased in either cohort and would not support microthrombi as the predominant reason for increased erythroid turnover. Alternatively, past work has shown that angiotensin II regulates normal erythropoiesis and stimulates early erythroid proliferation through unclear downstream mechanisms. SARS-CoV-2 binding to ACE2 may affect erythropoiesis through the downstream angiotensin II pathway. The significant increase in cfDNA derived from red blood progenitor cells, may alternatively be due to injury to red cell precursors, through direct or indirect processes.

What is claimed is:

1. A method for assessing the severity and progression of a SARS-CoV2 infection in a subject, comprising:
measuring the amount of total cell-free DNA (cfDNA) molecules in a biological sample from the subject, wherein an increased level of total cfDNA molecules from the sample as compared to a control level is indicative of increased severity and disease progression of SARS-CoV2 infection in the subject.

2. A method for assessing the likelihood to benefit from a selected clinical intervention given to a subject suffering from a SARS-CoV2 infection, comprising:
measuring the amount of total cell-free DNA (cfDNA) molecules in a biological sample from the subject, wherein an increased amount of total cfDNA molecules from the sample as compared to a control amount correlates with the likelihood to benefit from the selected clinical intervention.

3. The method of claim 1 or claim 2, wherein the measuring is achieved by an assay selected from quantitative PCR (qPCR), digital droplet PCR (ddPCR), a flourometric DNA quantification assay and a spectroscopic DNA quantification assay.

4. The method of any one of the preceding claims, wherein the biological sample is a blood sample, a serum sample, a plasma sample, a urine sample, a bronchoalveolar lavage sample, or a saliva sample.

5. The method of any one of the preceding claims, wherein the subject is monitored by measuring the amount of total cfDNA molecules periodically.

6. The method of any one of the preceding claims, wherein the increased severity and progression of SARS-CoV2 infection is reflected by admission to an intensive care unit and/or need for mechanical ventilation.

7. The method of any one of the preceding claims, further comprising providing a therapeutic regimen to the subject based on the result of the assessment.

8. The method of any one of claims 2-7, wherein the selected clinical intervention comprises administration of one or more of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant.

9. The method of claim 8, wherein the antiviral agent comprises remdesivir.

10. The method of claim 8, wherein the steroid comprises dexamethasone.

11. The method of claim 8, wherein the clinical intervention comprises one or more of mechanical ventilation and oxygen supplementation.

12. A method for assessing the severity and progression of a SARS-CoV2 infection in a subject comprising:
obtaining cell-free DNA (cfDNA) molecules from a biological sample from the subject;
measuring the amount of total cfDNA molecules in the biological sample,
measuring the amount of tissue-specific cfDNA molecules in the biological sample, wherein the tissue-specific cfDNA molecules come from a tissue selected from the group consisting of erythroblast, kidney and liver; and
determining the fraction of the amount of tissue-specific cfDNA molecules relative to the amount of total cfDNA molecules; wherein an increased fraction relative to a control is indicative of increased severity and disease progression in the subject.

13. The method of claim 12, wherein an increased fraction relative to a control is indicative of a high risk for mortality.

14. The method of claim 12 or 13, wherein the biological sample is a blood sample, a serum sample, a plasma sample, a urine sample, a bronchoalveolar lavage sample, or a saliva sample.

15. The method of any one of claims 12-14, wherein the subject is monitored by measuring the amount of total and tissue-specific cfDNA molecules periodically.

16. The method of any one of claims 12-15, wherein further comprising providing a therapeutic regimen to the subject based on the result of the assessment.

17. The method of any one of claims 12-16, wherein the measuring of the amount of tissue-specific cfDNA comprises:
determining the profiles of an epigenetic marker within the cfDNA molecules, wherein the epigenetic marker displays tissue-specific profiles; and
identifying the tissues of origin of the cfDNA molecules based on the profiles determined.

18. The method of claim 17, wherein the epigenetic marker is selected from the group consisting of a DNA modification, a histone modification, and nucleosome positioning.

19. The method of claim 18, wherein the DNA modification is DNA methylation or DNA hydroxymethylation.

20. The method of claim 18, wherein the histone modification is selected from the group consisting of acetylation, methylation, phosphorylation, ubiquitylation, GlcNAcylation, citrullination, krotonilation, and isomerization.

21. The method of claim 17, wherein the determining the profiles of the epigenetic marker comprises determining the sequences of the cfDNA molecules.

22. The method of claim 19, wherein the profile of DNA methylation is determined by bisulfite treatment or enzymatic DNA methylation analysis.

23. The method of claim 19, wherein the profile of DNA hydroxymethylation is determined by a pull down assay, a selective labeling assay, or an oxidative bisulfite sequencing assay.

24. The method of claim 18, wherein the profile of histone modification is detected by a pull-down assay.

25. The method of claim 18, wherein the nucleosome positioning is determined by a nucleosome positioning assay.

26. The method of claim 17, wherein the determining the profiles of the epigenetic marker is achieved without determining the sequences of the cfDNA molecules.

27. The method of claim 18, wherein the determining is achieved by a PCR assay selected from quantitiative PCR (qPCR) and digital droplet PCR (ddPCR).

28. The method of claim 27, wherein the assay comprises amplifying cfDNA molecules from regions of the genome that have specific epigenetic markers.

29. A method for detecting tissue damage in a subject suffering from a SARS-CoV2 infection comprising:
obtaining cfDNA molecules from a biological sample from the subject;
determining the profiles of an epigenetic marker within the cfDNA molecules, wherein the epigenetic marker displays tissue-specific profiles;
identifying the tissues of origin of the cfDNA molecules based on the profiles determined; and
measuring the level of cfDNA molecules from an identified tissue of origin, wherein (i) the level or (ii) an increased level of cfDNA molecules from said identified tissue of origin as compared to a control level, is indicative of damage in said identified tissue of origin.

30. The method of claim 29, wherein the epigenetic marker is selected from the group consisting of a DNA modification, a histone modification, and nucleosome positioning.

31. The method of claim 30, wherein the DNA modification is DNA methylation or DNA hydroxymethylation.

32. The method of claim 30, wherein the histone modification is selected from the group consisting of acetylation, methylation, phosphorylation, ubiquitylation, GlcNAcylation, citrullination, krotonilation, and isomerization.

33. The method of claim 29, wherein the biological sample is a blood sample, a serum sample, a plasma sample, a urine sample, a bronchoalveolar lavage sample, or a saliva sample.

34. The method of claim 29, wherein the tissues of origin comprise a solid organ.

35. The method of claim 34, wherein the solid organ is an organ selected from kidney, liver, spleen, and pancreas.

36. The method of claim 29, wherein the cfDNA molecules are from one or more organs selected from skin, heart, kidney, liver, lungs, stomach, bladder or pancreas.

37. A method for treating a SARS-CoV2 infection in a subject, comprising:
measuring the amount of total cell-free DNA (cfDNA) molecules in a biological sample from the subject; and
treating the subject with a clinical intervention or adjusting the ongoing clinical intervention when an increased level of total cfDNA molecules from the sample as compared to a control level is measured.

38. The method of claim 37, wherein the selected clinical intervention comprises administration of one or more of a steroid, an antiviral agent, a non-steroidal anti-inflammatory drug (NSAID), an ACE inhibitor, an angiotensin receptor blocker, convalescent plasma, an antibiotic, Interferon β, tocilizumab, and an anticoagulant.

39. The method of claim 38, wherein the antiviral comprises remdesivir.

40. The method of claim 38, wherein the steroid comprises dexamethasone.

41. The method of claim 38, wherein the clinical intervention comprises one or more of mechanical ventilation and oxygen supplementation.

42. The method of any one of claims 37-41, wherein the measuring is achieved by an assay selected from quantitative PCR (qPCR), digital droplet PCR (ddPCR), a flourometric DNA quantification assay and a spectroscopic DNA quantification assay.

43. The method of any one of claims 37-42, wherein the biological sample is a blood sample, a serum sample, a plasma sample, a urine sample, a bronchoalveolar lavage sample, or a saliva sample.

44. The method of any one of claims 37-43, wherein the subject is monitored by measuring the amount of total cfDNA molecules periodically.

45. A method for treating a SARS-CoV2 infection in a subject comprising:
obtaining cell-free DNA (cfDNA) molecules from a biological sample from the subject;
measuring the amount of total cfDNA molecules in the biological sample;
measuring the amount of tissue-specific cfDNA molecules in the biological sample, wherein the tissue-specific cfDNA molecules come from a tissue selected from the group consisting of erythroblast, kidney and liver;
determining the fraction of the amount of tissue-specific cfDNA molecules relative to the amount of total cfDNA molecules; and
treating the subject with a clinical intervention or adjusting the ongoing clinical intervention when the fraction of the amount of tissue-specific cfDNA molecules is increased relative to a control.

46. The method of claim 45, wherein an increased fraction relative to a control is indicative of a high risk for mortality.

47. The method of claim 45 or claim 46, wherein the biological sample is a blood sample, a serum sample, a plasma sample, a urine sample, a bronchoalveolar lavage sample, or a saliva sample.

48. The method of any one of claims 45-47, wherein the subject is monitored by measuring the amount of total and tissue-specific cfDNA molecules periodically.

49. The method of any one of claims 45-48, wherein the measuring of the amount of tissue-specific cfDNA comprises:
determining the profiles of an epigenetic marker within the cfDNA molecules, wherein the epigenetic marker displays tissue-specific profiles; and
identifying the tissues of origin of the cfDNA molecules based on the profiles determined.

50. A method of detecting a microbial co-infection of a subject suffering from a SARS-CoV2 infection comprising:
obtaining cell-free DNA (cfDNA) molecules from a biological sample from the subject;
determining the sequences of the cfDNA molecules; and
identifying the presence of a cfDNA sequence of a microbial species other than SARS-CoV2, thereby detecting a co-infection by the microbial species.

51. The method of claim 50, wherein the extracted cfDNA molecules are bisulfite treated before determining the sequences of the cfDNA molecules.

52. The method of claim 50 or claim 51, further comprising treating the subject with an anti-microbial agent when a microbial cfDNA sequence is identified in the biological sample.

53. The method of claim 52, wherein the anti-microbial agent is an anti-bacterial or anti-fungal agent.

54. The method of claim 53, wherein the anti-microbial agent is an anti-viral agent.

55. The method of any one of claims 50-54, wherein the biological sample is a blood sample, a serum sample, a plasma sample, a urine sample, a bronchoalveolar lavage sample, or a saliva sample.
